(19) United States
(12) Patent Application Publication
(10) Pub. No.: US 2005/0021037 A1
(43) Pub. Date: Jan. 27, 2005

(54) Image-guided navigated precision reamers

(76) Inventors: Daniel L. McCombs, Germantown, TN (US); Chester Paul Wheeler, Hernando, MS (US)

Correspondence Address: CHIEF PATENT COUNSEL;SMITH & NEPHEW, INC., 1450 BROOKS ROAD, MEMPHIS, TN, 38116, US

(21) Appl. No.: 10/857,535

(22) Filed: May 28, 2004

Related U.S. Application Data
(60) Provisional application No. 60/474,178, filed on May 29, 2003.

(51) Int. Cl. A61B 17/16

(52) U.S. Cl. 606/79

(57) ABSTRACT

Methods and systems for providing an image guided navigated precision reamer are set forth. According to one embodiment, a method comprising providing a reamer device, the reamer device comprising a reamer shaft adapted to provide a locking capability for receiving a reamer component and capable of being rotated in use, an intermediate sleeve adapted to receive the reamer shaft substantially coaxially, and an outer sleeve adapted to receive the intermediate sleeve, wherein the reamer shaft is adapted to rotate relative to the outer sleeve without translation relative to the outer sleeve, and the sleeve is adapted to function as a handle during reaming; providing a sensor apparatus for sensing position and orientation of a plurality of location indicia; providing a first set of location indicia coupled to an anatomical reference; providing a second set of location indicia coupled to the reamer device; providing a prosthetic implant; referencing information obtained from the first and second set of location indicia; reaming a cavity of an osteological anatomical structure in preparation for receiving the prosthetic implant based at least in part on the referenced information; and placing the prosthetic implant into the cavity is set forth.

### BACKGROUND OF THE INVENTION

1. Field of the Invention

The invention is directed to navigated reamers. More specifically, products and methods for preparing a patient's bone for receiving various types of prostheses with the aid of an image guided reamer are disclosed.

2. Description of the Related Art

Prosthetic joint replacement implants, including hip joints, shoulder joints and knee joints, are widely used in orthopedic surgery. Both artificial hip joints and artificial shoulder joints are generally ball and socket joints, designed to match as closely as possible the function of the natural joint. Generally, the artificial socket is implanted in one bone, and the artificial ball articulates in the socket. A stem structure attached to the ball is implanted in another of the patient's bones, securing the ball in position.

The ball and socket joint of the human hip unites the femur to the pelvis, wherein a ball-shaped proximal end of the femur is positioned within a socket-shaped acetabulum of the innominate bone of the pelvis. The proximal end or head of the femur acts as a ball fitting into the acetabulum socket, forming a ball and socket joint which allows the lower limb to move through flexion, extension, adduction, abduction, and rotation in a wide range or motion. The acetabulum is lined with cartilage, which cushions the femur and innominate bones and allows the joint to rotate smoothly with minimal friction. An envelope of tough ligaments connects the pelvis and femur, covering the joint and stabilizing it. Cartilage also makes the joint strong enough to support the weight of the upper body and resilient enough to absorb the impact of exercise and activity. A healthy hip allows the lower limb to move freely within its range of motion, while supporting the upper body and absorbing the impact that accompanies certain activities.

However, various degenerative diseases and injuries may require replacement of all or a portion of a hip using synthetic materials. Prosthetic components are generally made from either metals, ceramics, or plastics.

Total hip arthroplasty and hemi-arthroplasty are two procedures well known within the medical industry for replacing all or part of a patient's hip and have enabled hundreds of thousands of people to live fuller, more active lives. A total hip arthroplasty replaces both the femoral component and the acetabular surface of the joint, so that both a femoral prosthesis and an acetabular prosthesis are required. A hemi-arthroplasty may replace either the femoral component or the acetabular surface of the joint. The purpose of hip replacement surgery is to remove the damaged and worn parts of the hip and replaces them with artificial parts, called prostheses, which will help make the hip strong, stable and flexible again.

To duplicate the hip joint's natural action, a total hip replacement implant has three parts: the stem, which fits into the femur and provides stability; the ball, which replaces the spherical head of the femur; and the cup, which replaces the worn-out hip socket.

A conventional acetabular prosthesis may include a cup, a cup and a liner, or in some cases only a liner, all of which may be formed in various shapes and sizes. Generally, a metal cup and a polymeric liner are used. However, the liner may be made of a variety of materials, including polyethylene, ultra high molecular weight polyethylene and ceramic materials. The cup is usually of generally hemispherical shape and features an outer, convex surface and an inner, concave surface that is adapted to receive a cup liner. The liner fits inside the cup and has a convex and concave surface. The cup liner is the bearing element in the acetabular component assembly. The convex surface of the liner corresponds to the inner concave surface of the cup or acetabulum, and the liner concave surface receives the head of a femoral component. An acetabular cup may include a highly polished inner surface in order to decrease wear.

The stem and ball portion of the prosthesis may be a femoral prosthesis that generally includes a spherical or near-spherical head attached to an elongate stem with a neck connecting the head and stem. In use, the elongate stem is located in the intramedullary canal of the femur and the spherical or near-spherical head articulates relative to the acetabular component. Femoral prostheses used in total hip arthroplasty procedures may or may not differ from an endoprosthesis used in a hemi-arthroplasty. The femoral head of each type prosthesis is generally a standard size and shape. Various cups, liners, shells, stems and other components may be provided in each type arthroplasty to form modular prostheses to restore function of the hip joint.

During a total hip replacement, the surgeon will take a number of measurements to ensure proper prosthesis selection, limb length and hip rotation. After making the incision, the surgeon works between the large hip muscles to gain access to the joint. The femur is pushed out of the socket, exposing the joint cavity. The deteriorated femoral head is removed and the acetabulum is prepared by cleaning and enlarging with circular reamers of gradually increasing size. The new acetabular shell is implanted securely within the prepared hemispherical socket. The plastic inner portion of the implant is placed within the metal shell and fixed into place.

Current reamers that are provided for preparing a patient's hip socket to receive an acetabular cup typically feature a reamer handle, which cooperates with a reamer dome. The reamer handle may comprise a Teflon sleeve which houses a reamer shaft. The Teflon sleeve provides a grip for the surgeon and the reamer shaft with the reamer dome attached can be maneuvered back and forth within the sleeve as necessary for preparation of the bone. The fit between the shaft and the sleeve is not tight, but the shaft is freely movable with the sleeve.

Such reamers are used “free-hand” by the surgeon. In other words, the surgeon does not use any device for navigating the location of the reamer other than hand-eye coordination.

Next, the femur is prepared to receive the stem. The hollow center portion of the bone is cleaned and enlarged, creating a cavity that matches the shape of the implant stem. The top end of the femur is planed and smoothed so the stem can be inserted flush with the bone surface. If the ball is a separate piece, the proper size is selected and attached. Finally, the ball is seated within the cup so the joint is properly aligned and the incision is closed.

The ball and socket joint of the human shoulder is prepared using a procedure similar to that described above. During a shoulder replacement operation, at least a portion of the proximal section of the humeral shaft is replaced by a metal prosthesis. This prosthesis generally consists of two parts: a stem that is mounted into the medullary canal of the humerus, and a head component connected in some manner to the stem. The head component replaces the bearing surface of the humerus and articulates within the glenoid cavity of the scapula to allow movement of the shoulder.

An arthritic humeral head (ball of the joint) may be removed and replaced with a humeral prosthesis. If the glenoid socket is unaffected, a hemiarthroplasty may be performed (which means that only the ball is replaced). The humeral component is made of metal and is usually press fit, but sometimes cemented, into the shaft of the bone of the humerus.

If the glenoid is affected, but conditions do not favor the insertion of a glenoid component, a non-prosthetic glenoid arthroplasty may be performed along with a humeral hemiarthroplasty. In this procedure, the glenoid shape and orientation are corrected, but a glenoid prosthesis is not inserted. The socket can be reshaped using a spherical reamer. The prosthetic ball of the humeral component articulates with the reshaped bony socket of the glenoid.

In a total shoulder joint replacement, the glenoid bone is shaped and oriented with a spherical reamer, and then covered with a glenoid component. A small amount of bone cement is commonly used to hold the artificial glenoid socket in place. The reamers typically used are similar to those described above for use with the preparation of a hip socket for receiving an acetabular cup.

Reamers may also be used in connection with various trauma situations, for knee replacement surgeries, and so forth. Reamers may have a spherical dome if the cavity being shaped is spherical or may have an elongated shape if the cavity being shaped is a canal.

One advancement in joint replacement surgery has been improved surgical instrumentation and techniques for implanting various prostheses. For example, a leading cause of wear and revision in prosthetics such as knee implants, hip implants and shoulder implants is less than optimum implant alignment. In a total knee arthroplasty, for example, current instrument design for resection of bone limits the alignment of the femoral and tibial resections to average values for varus/valgus flexion/extension, and external/internal rotation. Additionally, surgeons often use visual landmarks or “rules of thumb” for alignment which can be misleading due to anatomical variability. Surgeons also rely on instrumentation to predict the appropriate implant size for the femur and tibia instead of the ability to intraoperatively template the appropriate size of the implants for optimal performance. Another challenge for surgeons is properly reaming and preparing the intramedullary canal or bone cavity to receive the implant. There are no instruments currently available that allow for precision reaming.

Once a bone has been resected, many of the visual landmarks are no longer present, making alignment and restoration of the joint line difficult. The present invention is applicable to repair, reconstruction or replacement surgery in connection with any other joint of the body.

Several manufactures currently produce image-guided surgical navigation systems that are used to assist in performing surgical procedures. The TREON™ and ION™ systems with FLUORONAV™ software manufactured by Medtronic Surgical Navigation Technologies, Inc. are examples of such systems. Systems and methods for accomplishing image-guided surgery are also disclosed in U.S. Ser. No. 10/364,859 filed Feb. 11, 2003 entitled “Image Guided Fracture Reduction,” which claims priority to U.S. Ser. No. 60/355,886 filed Feb. 11, 2002 entitled “Image Guided Fracture Reduction”; U.S. Ser. No. 60/271,818 filed Feb. 27, 2001, entitled “Image Guided System for Arthroplasty”; and U.S. Ser. No. 10/229,372 filed Aug. 27, 2002 entitled “Image Computer Assisted Knee Arthroplasty,” the entire contents of each of which are incorporated herein by reference as are all documents incorporated by reference therein. Further image-guided surgery devices, systems, and methods are disclosed in a provisional application entitled SURGICAL NAVIGATION SYSTEMS AND PROCESSES, Application Ser. No. 60/355,899, filed on Feb. 11, 2002, hereby incorporated by this reference.

Systems and processes according to one embodiment of the present invention use position and/or orientation tracking sensors such as infrared sensors acting stereoscopically or otherwise to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated fiducials or based on stored position and/or orientation information.

The processing functionality correlates this position and orientation information for each object with stored information regarding the items, such as a computerized fluoroscopic imaged file of a femur, tibia, humerus, acetabulum, or glenoid cavity, a wire frame data file for rendering a representation of an instrumentation component, trial prosthesis or actual prosthesis, or a computer generated file relating to a rotational axis or other virtual construct or reference. The processing functionality then displays position and orientation of these objects on a screen or monitor, or otherwise. Thus, systems and processes according to one embodiment of the invention can display and otherwise output useful data relating to predicted or actual position and orientation of body parts, surgically related items, implants, and virtual constructs for use in navigation, assessment, and otherwise performing surgery or other operations.

As one example, images such as fluoroscopy images showing internal aspects of any bone or cavity can be displayed on the monitor in combination with actual or predicted shape, position and orientation of surgical implements, instrumentation components, trial implants, actual prosthetic components, and rotational axes in order to allow the surgeon to properly position and assess performance of various aspects of the joint being repaired, reconstructed or replaced. The surgeon may navigate tools, instrumentation, trial prostheses, actual prostheses and other items relative to bones and other body parts in order to perform the procedure more accurately, efficiently, and with better alignment and stability. Systems and processes according to the present invention can also use the position tracking information and, if desired, data relating to shape and configuration of surgical related items and virtual constructs or references in order to produce numerical data which may be used with or without graphic imaging to perform tasks such as assessing performance of trial prosthetics statically and throughout a range of motion, appropriately modifying tissue such as ligaments to improve such performance and similarly assessing performance of actual prosthetic components which have been placed in the patient for alignment and stability.

Systems and processes according to the present invention can also generate data based on position tracking and, if desired, other information to provide cues on screen, aurally or as otherwise desired to assist in the surgery such as suggesting certain bone modification steps or measures which may be taken to release certain ligaments or portions of them based on performance of components as sensed by systems and processes according to the present invention.

For example, the Medtronic systems referred to above use fluoroscopic imaging to capture anatomical characteristics and infrared cameras that detect certain targets placed in the surgical field to track instruments and anatomy. As used herein, an infrared camera can be any type of sensor or detector that is capable of sensing or detecting light of an infrared wavelength. Any number and orientation of so-called targets, fiducials, frames, markers, indicia, or any other desired location-assisting functionality (“references”) can be used as targets to be detected by an imaging system or sensor.

Other imaging or data capture systems such as CT, MRI, visual, sonic, digitized modeling, traditional x-ray equipment, or any other effective system or technique which has the capacity to image bone or other desired structures or tissue in the body can be used. Such systems generally include transducer functionality for emitting energy or otherwise performing sensing or location of objects and anatomical structure, a processor, mass memory storage, input/output functionality to control and direct operation of the system, and at least one monitor or other visual output functionality for rendering images that may be constructed by the system, whether or not in combination with images obtained from the transducer in real time.

Such systems typically combine processes and functionality for obtaining, storing, manipulating and rendering images of internal body structure with functionality that senses, stores, manipulates and virtually renders representations of components or objects such as instrumentation, trial components, surgical tools and other objects. The systems can then generate and display representations of the objects in combination with images of the body structure or tissue.

Such combination renderings can be created using real time imaging of the body structure or tissue, or the system can obtain appropriate imaging of such structure or tissue and later computer generate and display renderings of it. The Medtronic systems, for instance, require the use of references attached to the anatomy, typically in a substantially rigid fashion, such as to bone structure. The system tracks movement of the reference in three dimensions and then generates images of the bone structure's corresponding motion and location.

The references on the anatomy and the instruments either emit or reflect infrared light that is then detected by an infrared camera. The references may be sensed actively or passively by infrared, visual, sound, magnetic, electromagnetic, x-ray, or any other desired technique. An active reference emits energy, and a passive reference merely reflects energy. In some embodiments, the references have at least three, but usually four, markers that are tracked by an infrared sensor to determine the orientation of the reference and thus the geometry of the instrument, implant component or other object to which the reference is attached. References have been attached to surgical and implant devices such as instrumentation, trial instruments, and the like. For example, references have been attached to probes, instruments for placing acetabular cups and trial implants, drill guides, and cutting blocks.

The Medtronic imaging systems allow references to be detected at the same time the fluoroscopy imaging is occurring. Therefore, the position and orientation of the references may be coordinated with the fluoroscope imaging. Then, after processing position and orientation data, the references may be used to track the position and orientation of anatomical features that were recorded fluoroscopically. Computer-generated images of instrumentation, components, or other structures that are fitted with references may be superimposed on the fluoroscopic images. The instrumentation, trial, implant or other structure or geometry can be displayed as 3-D models, outline models, or bone-implant interface surfaces.

Current systems and techniques do not provide for effective image-guided navigated reaming of various bone cavities. For instance, systems which use CT and MRI data generally require the placement of reference frames pre-operatively which can lead to infection at the pin site. The resulting 3D images must then be registered, or calibrated, to the patient anatomy intraoperatively. Current registration methods are less accurate than the fluoroscopic system. These imaging modalities are also more expensive. Some “imageless” systems, or non-imaging systems, require digitizing a large number of points to define the complex anatomical geometries of the knee at each desired site. This can be very time intensive resulting in longer operating room time. Other imageless systems determine the mechanical axis of the knee by performing an intraoperative kinematic motion to determine the center of rotation at the hip, knee, and ankle. This requires placement of reference frames at the iliac crest of the pelvis and in or on the ankle. This calculation is also time consuming at the system must find multiple points in different planes in order to find the center of rotation. This is also problematic in patients with a pathologic condition. Ligaments and soft tissues in the arthritic patient are not normal and thus will give a center of rotation that is not desirable for normal knees. Robotic systems require expensive CT or MRI scans and also require pre-operative placement of reference frames, usually the day before surgery. These systems are also much slower, almost doubling operating room time and expense.

None of these systems can effectively track bone position during a range of motion and calculate the relative positions of the articular surfaces, the placement of instrumentation such as reamers with respect to the bone, among other things. Also, none of them currently make suggestions on ligament balancing, display ligament balancing techniques, or surgical techniques.

Improved products and methods would include structures and techniques for guiding a reamer so that precise and precision reaming may be accomplished. Improved products and methods would also provide for reduced numbers of x-ray, fluoroscopic, or other images, and would not necessitate pre-operative imaging or surgical procedures prior to the primary procedure.

### SUMMARY

An embodiment according to certain aspects of the invention is a device for precision reaming a cavity or canal for receiving a prosthesis. Another embodiment is a method of precision reaming a cavity or canal for receiving a prosthesis using an image guided navigation system.

### BRIEF DESCRIPTION OF THE DRAWINGS

FIG. 1 is an exploded perspective view of a navigated reamer according to certain aspects of the invention.

FIG. 2a is a side plan view of a reamer shaft of the navigated reamer of FIG. 1.

FIG. 2b is a side exploded view of a reamer shaft of the navigated reamer of FIG. 1.

FIG. 2c is a side plan view of an assembled reamer shaft.

FIG. 3 is a side plan and perspective view of an intermediate sleeve of the navigated reamer of FIG. 1.

FIG. 4 is a perspective view of an outer sleeve of the navigated reamer of FIG. 1.

FIG. 5 is a perspective schematic view showing a navigated reamer being positioned with respect to a bone.

### DETAILED DESCRIPTION

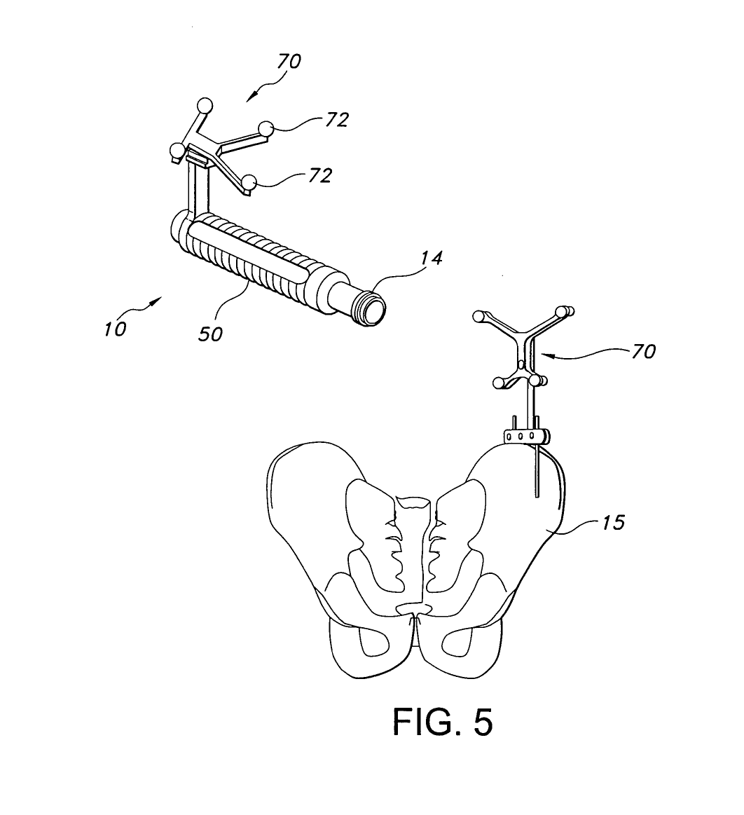

Systems and processes according to certain embodiments of the present invention use computer capacity, including standalone and/or networked computers, to store data regarding spatial aspects of surgically related items and virtual constructs or references including body parts, implements, instrumentation, trial components, prosthetic components and rotational axes of body parts. Any or all of these may be physically or virtually connected to or incorporate any desired form of mark, structure, component, or other fiducial or reference device or technique which allows position and/or orientation of the item to which it is attached to be sensed and tracked, preferably in three dimensions of translation and three degrees of rotation as well as in time if desired. In one embodiment, such “fiducials” or “references” are reference frames, each containing at least three, preferably four, sometimes more, reflective elements such as spheres reflective of lightwave or infrared energy, or active elements such as LEDs. An exemplary reference is shown by FIG. 5.

Orientation of the elements on a particular fiducial or reference may vary from one fiducial to the next so that sensors according to the present invention may distinguish between various components to which the fiducials are attached in order to correlate for display and other purposes data files or images of the components. Some fiducials use reflective elements and some use active elements, both of which may be tracked by preferably two, sometimes more infrared sensors whose output may be processed in concert to geometrically calculate position and orientation of the item to which the fiducial is attached.

Position/orientation tracking sensors and fiducials need not be confined to the infrared spectrum. Any electromagnetic, electrostatic, light, sound, radiofrequency or other desired technique may be used. Alternatively, each item such as a surgical implement, instrumentation component, trial component, implant component or other device may contain its own “active” fiducial such as a microchip with appropriate field sensing or position/orientation sensing functionality and communications link such as spread spectrum RF link, in order to report position and orientation of the item. Such active fiducials, or hybrid active/passive fiducials such as transponders can be implanted in the body parts or in any of the surgically related devices mentioned above, or conveniently located at their surface or otherwise as desired. Fiducials may also take the form of conventional structures such as a screw driven into a bone, or any other three dimensional item attached to another item, position and orientation of such three dimensional item able to be tracked in order to track position and orientation of body parts and surgically related items. Hybrid fiducials may be partly passive, partly active such as inductive components or transponders which respond with a certain signal or data set when queried by sensors according to the present invention.

Systems and processes according to certain embodiments of the present invention employ a computer to calculate and store reference axes of body components. From these axes such systems track the position of the instrumentation and osteotomy guides so that bone resections and reaming will locate the implant position optimally, usually aligned with the mechanical axis. Furthermore, during trial reduction of the joint, the systems provide feedback on the balancing of the ligaments in a range of motion and under varus/valgus, anterior/posterior and rotary stresses and can suggest or at least provide more accurate information than in the past about which ligaments the surgeon should release in order to obtain correct balancing, alignment and stability. More particularly, the systems provide feedback on the depth of reaming and the position of the reamer with respect to the joint.

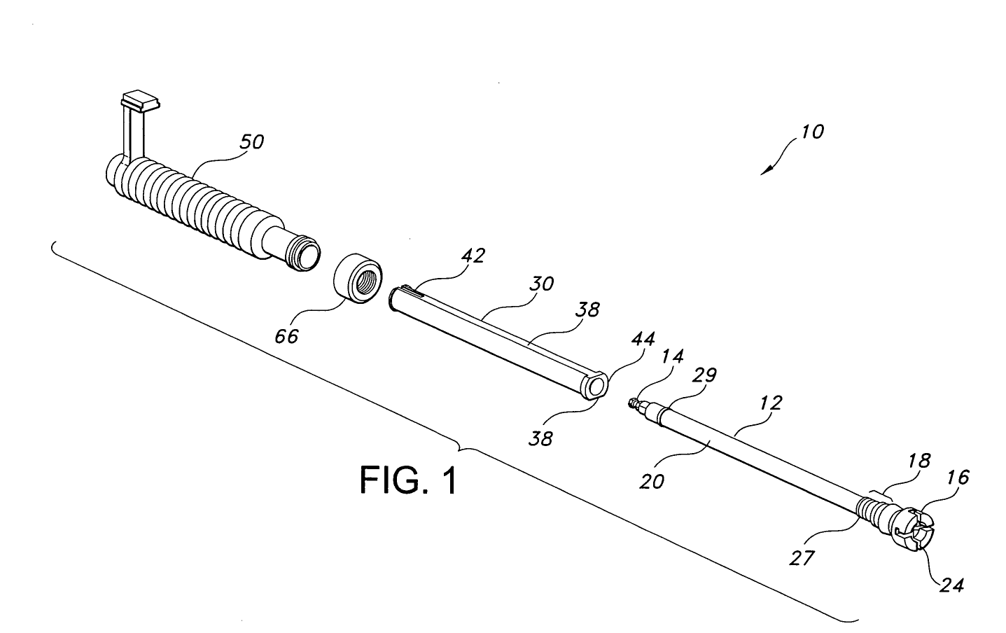

FIG. 1 shows a navigated reamer 10 according to certain aspects of the invention operable with an image-guided surgical navigation system. As described above, an image-guided surgical navigation system can be any of a variety of systems that capture anatomical characteristics and/or other references connected to the body and/or other surgical devices and/or other structures associated with a reference. Such a system then tracks parts of the body and the surgical devices relative to one another. Generally, reference to a system as “image-guided” means that the system produces images by which surgical navigation information is conveyed to the user. For example, a computer display showing virtual representations of an instrument and its relationship with a bone is considered one example of an image-guided system.

As shown in FIG. 5, the position and orientation of navigated reamer 10 (shown as a schematic) are recorded by placing navigated reamer end 14 on a portion of a reference 70 attached to a bone 15. Bone 15 may be an acetabular cup, the glenoid cavity, the femur, the tibia, the intramedullary canal, or any other appropriate anatomical reference point or even a non-anatomical reference point. The navigated reamer 10 is then used to prepare the bone to receive a prosthesis. As shown in FIG. 1, navigated reamer 10 includes three major elements: a reamer shaft 12, an intermediate sleeve 30, and an outer sleeve 50.

Reamer Shaft

The reamer shaft 12 includes a shank 20 with a head 16 designed to receive a reamer component and an annular locking component 18. The locking component 18 is mounted so that it can slide about the shank 20 and under the head 16. The reamer shaft is equipped with a locking mechanism which cooperates with the head 16 in order to lock the reamer component on the head 16.

Any shaft that provides a locking capability for receiving a reamer component and the capability to be rotated in use can be used in connection with the present invention. For the sake of completeness, an exemplary reamer shaft is shown and described, although it is understood that this reamer shaft description is not intended to be limiting or to exclude other possible reamer shafts from the scope of this invention.

An exemplary reamer shaft is manufactured by Precimed and described in U.S. Pat. No. 6,540,739, hereby incorporated herein by this reference. Specifically, the reamer shaft 12 has a cylindrical shank 20 and a head 16, similar to that described in U.S. Pat. No. 5,658,290, hereby incorporated herein by this reference. The head portion ends at neck 27, which provides a ledge from which shank 20 extends.

The head 16 has a central recess 22 and forms a crown around this recess 22. The crown has four retractable catches 24 diametrically opposite in pairs. A reamer component (not shown), for example, a reamer component similar to the one shown and described in U.S. Pat. No. 5,658,290, is able to be fixed in catches 24.

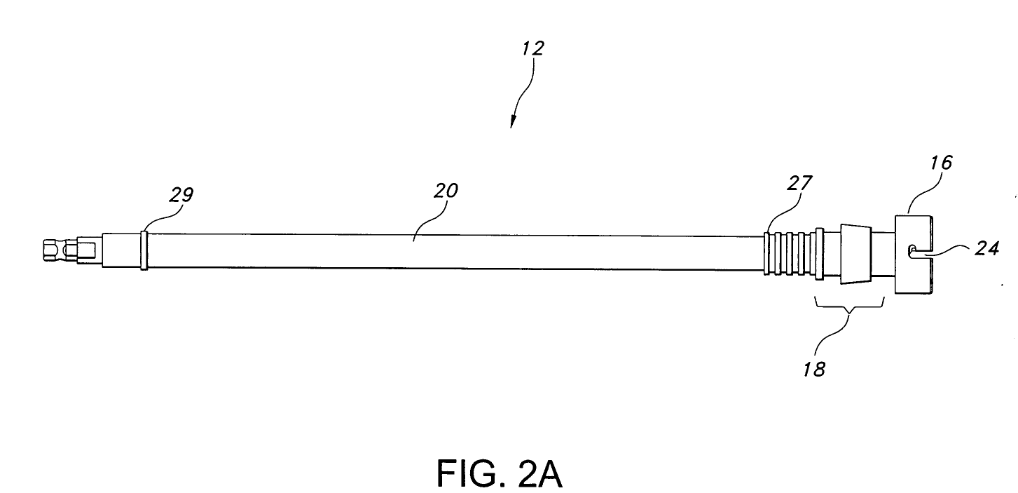

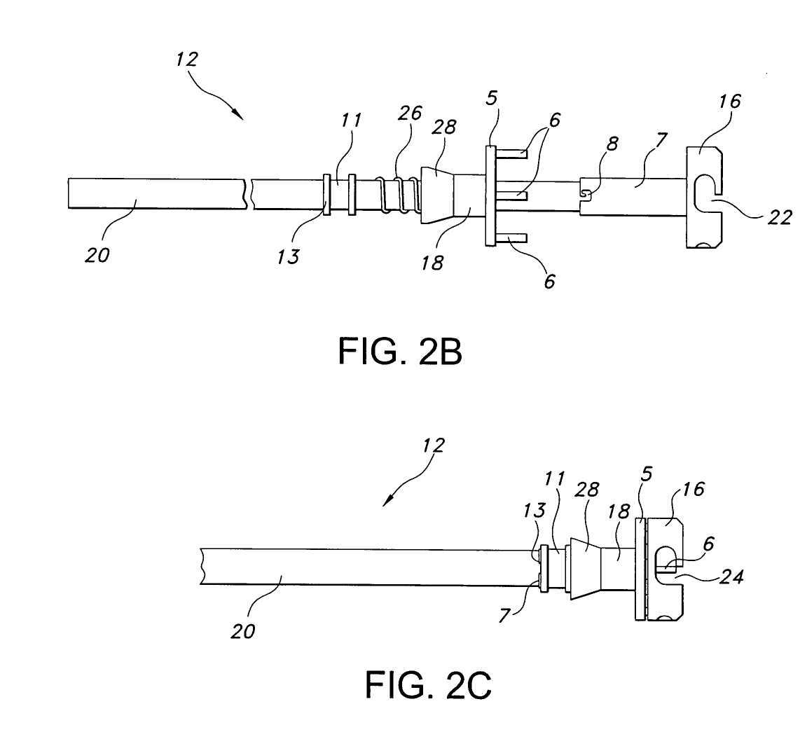

The reamer component is locked in the catches 24 by an annular locking component 18. As shown by FIG. 2b, the locking component 18 is equipped with a plate 5 having parallel fingers 6 (preferably four fingers 6) which pass through corresponding holes in the head 16 in order to close the catches 24, as is described in U.S. Pat. No. 5,658,290.

In certain embodiments, the locking component 18 does not slide directly on the section of the shank 20 seen in FIG. 2b, but on a section 7 with a greater diameter than the diameter of the rest of the shank. This section 7 can consist of a tubular component arranged on the shank 20. Also arranged around section 7 is a helical spring 26 which engages a frustoconical widened part 28 of the locking component 18 and bears against the locking component. The reamer shaft 12 is completed by a ring 11 which also slides on the section 7 and is equipped internally with a radial stud 13, that is oriented in the direction of the shank 20.

Starting from the disassembled position shown in FIG. 2b, and in order to assemble the reamer shaft 12, the locking component 18 is brought under the head 16, engaging its locking fingers 6 through the head, and the ring 11 pushes spring 26 against the locking component 18, compressing the spring 26 and at the same time turning the ring 11 to the left until its stud 13 engages in a catch. The reamer shaft 12 can then be used as is described in U.S. Pat. No. 5,658,290. The frustoconical widened part 28 gives a grip for the thumb and index finger for pulling the locking component 18 back counter to the action of the spring 26 in order to release the reamer component fixed on the reamer shaft.

The end of the shank remote from the head 16 is shown as having a hexagonal cross section for fastening the reamer shaft on a driving component for driving the reamer shaft in rotation. Any shape may be provided, as long as it can interface with a driving component, or can be maneuvered by hand, if appropriate. The end of shank remote from head also has a lip 29 for receiving and securing intermediate sleeve 30.

Intermediate Sleeve

As shown in FIG. 1, the intermediate sleeve 30 is tubular or cannulated. It has an inner diameter that is just slightly larger than the outer diameter of the reamer shaft 12, such that the intermediate sleeve 30 is adapted to receive the reamer shaft 12. The Intermediate sleeve 30 is desirably any material that allows for at least partial flexibility. On certain embodiments, sleeve 30 is Teflon or plastic.

Although intermediate sleeve is primarily tubular, in certain embodiments it has two flat portions 38 that run the length of sleeve 30. Flat portions 38 allow intermediate sleeve 30 to be received by outer sleeve 50 more easily than if it were completely tubular. They also allow navigated reamer 10 to be autoclaved or otherwise sterilized without its complete disassembly. The flat portions provide a small “D-shaped” opening between intermediate sleeve 30 and outer sleeve 50 when navigated reamer 10 is assembled, which will allow for complete sterilization.

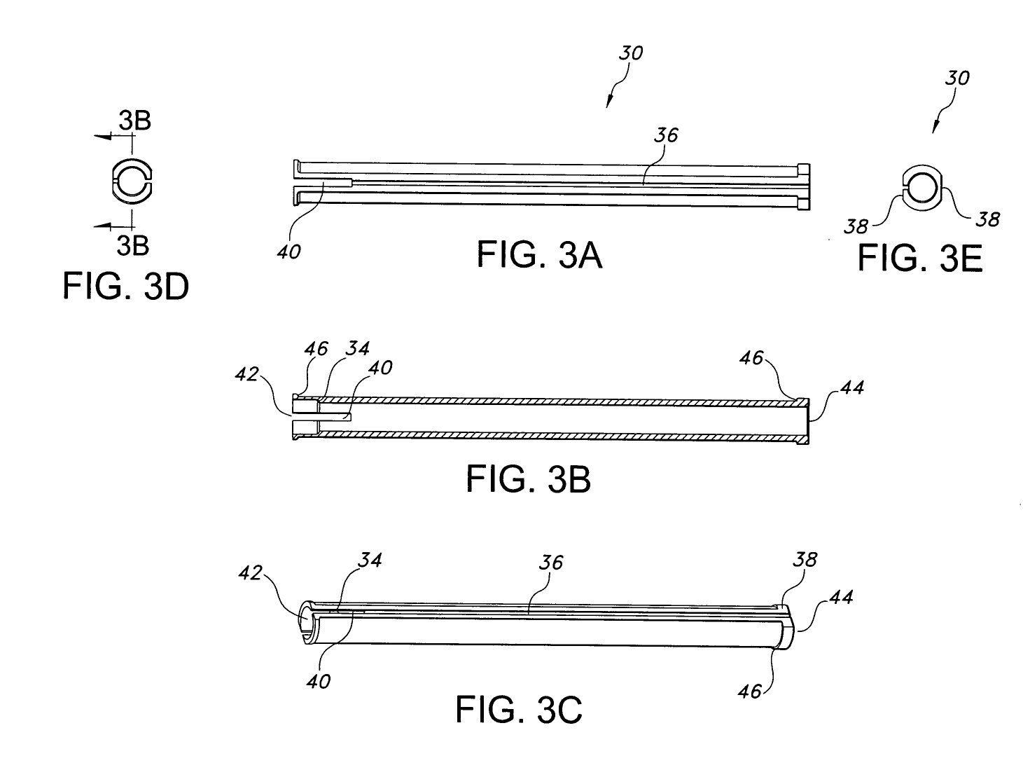

As shown more clearly by FIG. 3, intermediate sleeve 30 also has a throughslot 36 that runs the length of sleeve 30. Throughslot 36 may be located at one of the flat portions 38. Throughslot is provided so that the diameter of sleeve 30 can be compressed or constricted by the outer sleeve 50 when outer sleeve 50 is disposed over the intermediate sleeve 30 in use. The throughslot 36 also enables the intermediate sleeve to expanded as it slides over reamer shaft 12. In use, the throughslot 36 can allow sleeve 30 to expand slightly as it slides over reamer shaft 12 because the flexibility of sleeve 30 allows sleeve 30 to open slightly at throughslot 36 due to the pressure from reamer shaft 12 against the inner portion of sleeve 30. Intermediate sleeve 30 can then constrict around and tightly grip the reamer shaft 12 as outer sleeve 50 slides over intermediate sleeve 30. The tight fit between the out sleeve 50, intermediate sleeve 30, and reamer shaft 12 reduces translation between the reamer shaft 12 and the outer sleeve 50 while still allowing rotation of the reamer shaft relative to the outer sleeve.

Other optional features of intermediate sleeve include partial slits 40 near end 42. Partial slits allow sleeve 30 to expand even further as it slides over reamer shaft 12, and particularly over the lip 29 of reamer shaft.

The inner diameter of sleeve 30 has an inner, indented lip 34 that cooperates with lip 29 of reamer shaft 12. As opposite end 44 of sleeve 30 engages end 14 of reamer shaft 12, throughslot 36 expands slightly to allow sleeve 30 to slide easily along shank 20 of reamer shaft 12. Once inner lip 34 engages lip 29, and opposite end 44 abuts neck 27, the sleeve is positioned on reamer shaft 12 with a tight fit.

End 42 and opposite end 44 of intermediate sleeve 30 each form a ledge 46. Ledge 46 acts to position outer sleeve 50 is close fit with intermediate sleeve 30.

Although shown as a single sleeve 30, in alternate embodiments, intermediate sleeve 30 may comprise a plurality of sleeves.

Outer Sleeve

As shown by FIG. 1, outer sleeve 50 is adapted to slide over intermediate sleeve 30. The inner diameter of outer sleeve 50 is just slightly larger than the outer diameter of intermediate sleeve 30, such that there is a close fit created between the two. As previously described, the throughslot 36 on intermediate sleeve 30 allows it to expand to slide over reamer shaft 12 yet compress tightly as outer sleeve 50 is placed thereover. Once in place, outer sleeve 50 constricts the intermediate sleeve 30 along the length of the reamer shaft or in specific bearing locations. This control allows for accurate placement of the mount 54 (described below) with respect to the reamer shaft.

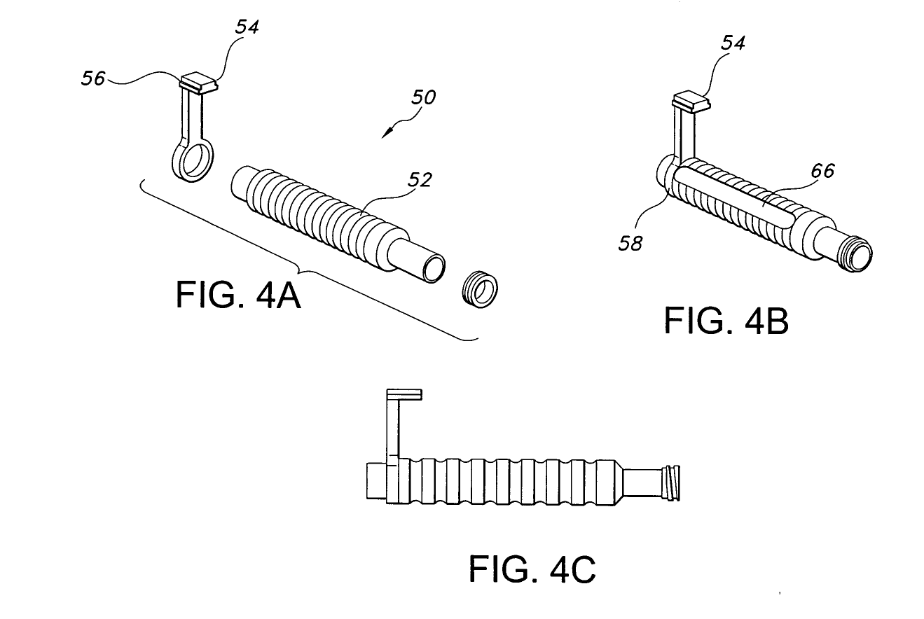

Outer sleeve 50 is illustrated by FIG. 4. It may be provided with grooves 52 in order to allow the surgeon to easily grip and apply force to the navigated reamer 10 without slipping. Outer sleeve 50 is used as a handle for the user in order to stead the navigated reamer during operation.

Outer sleeve may also have optional openings 60. In some embodiments, openings 60 extend at least part of the longitudinal length of outer sleeve 50. In certain embodiments, they are provided in order to assist with the cleaning of navigated reamer in its fully assembled position. For example, openings 60 allow navigated reamer to be autoclaved without being disassembled. Outer sleeve is substantially rigid and may be made from titanium, steel, or any other material that is suitable for surgical instruments.

Outer sleeve is also provided with mount 54, which is adapted to receive a reference 70 in order to allow for the image guidance described above. Reference 70 enables the navigated reamer 10 to be located by an image-guided surgical navigation system such that the precise position of navigated reamer and its attachments can be easily tracked. As illustrated in FIG. 5, reference 70 is coupled to the outer sleeve 50 in a predefined physical relationship. Various embodiments are shown by FIG. 4, in which mount 54 may be integral with outer sleeve 50 or it may be removable.

For the integral embodiment, a mount 54 is affixed at a specific location, for example, the proximal end 58 of the outer sleeve 50. A dovetail 56 is located at on the mount that is designed to be received by reference 70, which has corresponding mating dovetail opening (not shown).

Alternatively, mount 54 may be a bracket that is adapted to slide over proximal end 58 of outer sleeve 50. Although not shown, it is understood that the bracket may alternatively be a clamp that opens and closes to secure outer sleeve 50 or any other attachment device or structure suitable for attaching components to each other. Those skilled in the art will understand that any member that can attach reference 70 to navigated reamer 10 is considered a “bracket” or a “mount” within the scope of this invention.

Another embodiment of this invention provides a reference 70 having an integral attachment structure (not shown). Attachment structure may be a bracket integrally formed with reference 70 or any other connection element that will achieve securement of reference 70 to navigated reamer 10.

The aspect ratio of the outer sleeve 50 to the length of intermediate sleeve 30 is quite large. This large aspect ratio minimizes the angular error between the reamer shaft and the mount 54. Since the primary mode of measurement for the instrument is angular, this feature provides a distinct advantage over the prior art.

In order to lock the components in place once navigated reamer has been assembled, there may be provided a locking nut 66. Locking nut cooperates via threads, taper lock, or any other connecting mechanism with outer sleeve 50. In certain embodiments, locking nut 66 may slide over reamer shaft 12 before intermediate and outer sleeves. After the placement of intermediate sleeve 30, outer sleeve 50 is placed and locking nut 66 cooperates with threads, corresponding taper, or any other connecting mechanism that is provided on outer sleeve 50.

Systems and Methods

The invention may also be embodied in a system for reaming a portion of a patient's bone in order to prepare the bone to receive a prosthesis. The system is operable to virtually represent the position of the navigated reamer 10 with respect to the cavity or canal to be prepared. At least one segment of the bone is virtually represented with respect to a reference 70 and the navigated reamer 10 is also represented with respect to another reference 70.

The system includes a first reference coupled to the at least one bone segment, and a second reference coupled to the instrument. The first reference is coupled to a segment of bone toward which the instrument will be directed. In any case, the system also includes a detector operable to collect position and orientation information regarding the at least one segment and the instrument. As discussed in the background section above, the detector could be an infrared camera, visual camera, or any of a variety of sensors capable of detecting any kind of reference or characteristic. The system also includes a data processing device operable to store position and orientation information about one or more fractured segments and the instrument. The data processing device calculates virtual positions of the at least one fractured segment and the instrument based upon inputs from the detector. Such calculations could involve matrix transformations, table look-up functionality, or any other operation effective in calculating the respective virtual positions. An indicator device for notifying a user of the relative positions of the at least on fractured segment and the instrument is also provided. Such an indicator could be a visual cue on a computer screen such as color changes or alignment of articulating lines, sounds, flashes of light, or any device for showing a changeable condition, or some combination of any of these.

Another embodiment of the invention includes a method of reaming a cavity or canal of a bone into which a prosthesis is to be implanted. As shown in FIG. 5, one method includes attaching a first reference 70 to a segment of a bone. The position and orientation of reference 70 may then be recorded relative to a first datum. As used herein, the term “recording” includes without limitation capturing bbbcccor storing in computer memory or on a tangible medium such as film. Any such acquisition of information associated with position or orientation, regardless of how transiently maintained in a system, medium, or component is within the definition of recording as used herein. In some embodiments of the invention, recording may include the use of an infrared camera that registers the positions of energy-reflecting surfaces 72.

Alternatively, a reference may not be coupled with a segment of bone, but may be attached to a probe. Such a probe may be recorded at a predetermined anatomical position and orientation. Therefore, by knowing the position of the reference attached to the probe, and the probe's position and orientation on the anatomy, the position of the anatomy can be calculated.

Another reference is attached to a navigated reamer 10. As described above, the navigated reamer is operable to prepare a canal or cavity for receiving a prosthetic implant. As with the first reference, a position and orientation of the second reference is recorded.

Once all of the references, segments, and instrument (or instruments) have been located, they may all be continuously or intermittently tracked without the use of fluoroscopy for as long as desired. As used herein, “continuously” shall mean at a rate that appears substantially continuous to a user, but could include tracking accomplished at a standard electronic sampling rate such as a rate greater than one sample per second. Typically, this tracking is accomplished by use of a computer system that is interfaced with an infrared camera or other device, the computer also calculating transforms regarding each datum and its relationship to each other datum.

Therefore, embodiments of the invention provide for the location and tracking of bone segments and instruments such that the instruments may be used to prepare the patient to receive a prosthesis. This is accomplished with reduced numbers of x-ray, fluoroscopic, and other such energy-intense imaging devices. There is no requirement for pre-operative imaging or any surgical procedures prior to the primary procedure. With various embodiments of the invention, continuous or nearly continuous monitoring of bone segment and instrument positions is accomplished.

The particular embodiments of the invention have been described for clarity, but are not limiting of the present invention. Those of skill in the art can readily determine that additional embodiments and features of the invention are within the scope of the appended claims and equivalents thereto.

1. A surgical reamer navigation method comprising:
providing a reamer device, the reamer device comprising a reamer shaft adapted to provide a locking capability for receiving a reamer component and capable of being rotated in use, an intermediate sleeve adapted to receive the reamer shaft substantially coaxially, and an outer sleeve adapted to receive the intermediate sleeve, wherein the reamer shaft is adapted to rotate relative to the outer sleeve without translation relative to the outer sleeve, and the sleeve is adapted to function as a handle during reaming;
providing a sensor apparatus for sensing position and orientation of a plurality of location indicia;
providing a first set of location indicia coupled to an anatomical reference;
providing a second set of location indicia coupled to the outer sleeve;
providing a prosthetic implant;
referencing information obtained from the first and second set of location indicia;
reaming a cavity of an osteological anatomical structure in preparation for receiving the prosthetic implant based at least in part on the referenced information; and
placing the prosthetic implant into the cavity.

2. The method of claim 1, wherein the fit between the outer sleeve and the intermediate sleeve is sufficiently tight to minimize translation of the reamer shaft relative to the second set of location indicia.

3. The method of claim 2, wherein the outer sleeve covers a sufficient length of the reamer shaft to reduce non-rotational movement of the reamer shaft with respect to the outer sleeve.

4. The method of claim 1, wherein the reamer shaft comprises one or more retractable catches.

5. The method of claim 4, wherein the retractable catches are substantially diametrically opposed.

6. The method of claim 4, wherein the reamer component is locked in the intermediate catches by an annular locking component.

7. The method of claim 1, wherein the intermediate shaft is partially flexible.

8. The method of claim 1, wherein the intermediate shaft comprises a PTFE polymer.

9. The method of claim 1, wherein the intermediate shaft comprises plastic.

10. The method of claim 1, wherein the intermediate shaft comprises bushings or bearings.

11. The method of claim 1, wherein the intermediate shaft comprises one or more flat portions configured to facilitate reception by the outer sleeve.

12. The method of claim 1, wherein the intermediate shaft comprises openings configured to allow for complete sterilization.

13. The method of claim 1, wherein the intermediate shaft comprises a throughslot configured to allow the diameter of the intermediate sleeve to expand slightly when receiving the reamer shaft.

14. The method of claim 13, wherein the outer sleeve constricts a length of the intermediate sleeve tightly against the reamer shaft.

15. The method of claim 13, wherein the outer sleeve constricts the intermediate sleeve tightly against the reamer shaft at specific bearing locations.

16. A surgical reamer navigation system comprising:
a reamer device, the reamer device comprising a reamer shaft adapted to provide a locking capability for receiving a reamer component and capable of being rotated in use, an intermediate sleeve adapted to receive the reamer shaft substantially coaxially, and an outer sleeve adapted to receive the intermediate sleeve, wherein the reamer shaft is adapted to rotate relative to the outer sleeve without translation relative to the outer sleeve, and the sleeve is adapted to function as a handle during reaming;
a sensor apparatus for sensing position and orientation of a plurality of location indicia;
a first set of location indicia coupled to an anatomical reference;
a second set of location indicia coupled to the outer sleeve; and
a prosthetic implant.

17. The system of claim 16, wherein the fit between the outer sleeve and the intermediate sleeve is sufficiently tight to minimize translation of the reamer shaft relative to the second set of location indicia.

18. The system of claim 17, wherein the outer sleeve covers a sufficient length of the reamer shaft to reduce non-rotational movement of the reamer shaft with respect to the outer sleeve.

19. The system of claim 16, wherein the reamer shaft comprises one or more retractable catches.

20. The system of claim 19, wherein the retractable catches are substantially diametrically opposed.

21. The system of claim 19, wherein the reamer component is locked in the retractable catches by an annular locking component.

22. The system of claim 16, wherein the intermediate shaft is partially flexible.

23. The system of claim 16, wherein the intermediate shaft comprises a PTFE polymer.

24. The system of claim 16, wherein the intermediate shaft comprises plastic.

25. The system of claim 16, wherein the intermediate shaft comprises bushings or bearings.

26. The system of claim 16, wherein the intermediate shaft comprises one or more flat portions configured to facilitate reception by the outer sleeve.

27. The system of claim 16, wherein the intermediate shaft comprises openings configured to allow for complete sterilization.

28. The system of claim 16, wherein the intermediate shaft comprises a throughslot configured to allow the diameter of the intermediate sleeve to expand slightly when receiving the reamer shaft.

29. The system of claim 28, wherein the outer sleeve constricts the a length of the intermediate sleeve tightly against the reamer shaft.

30. The system of claim 28, wherein the outer sleeve constricts the intermediate sleeve tightly against the reamer shaft along specific bearing locations.

31. A surgical reamer navigation device comprising:
a reamer shaft adapted to provide a locking capability for receiving a reamer component and capable of being rotated in use;
an intermediate sleeve adapted to receive the reamer shaft substantially coaxially;
and an outer sleeve adapted to receive the intermediate sleeve, wherein the reamer shaft is adapted to rotate relative to the outer sleeve without translation relative to the outer sleeve, and the sleeve is adapted to function as a handle during reaming.

32. The device of claim 31, wherein the fit between the outer sleeve and the intermediate sleeve is sufficiently tight to minimize angular error between the reamer shaft and the second set of location indicia.

33. The device of claim 32, wherein the outer sleeve covers a sufficient length of the reamer shaft to reduce translation of the reamer shaft with respect to the outer sleeve.

34. The device of claim 31, wherein the reamer shaft comprises one or more retractable catches.

35. The device of claim 34, wherein the retractable catches are substantially diametrically opposed.

36. The device of claim 34, wherein the reamer component is locked in the retractable catches by an annular locking component.

37. The device of claim 31, wherein the intermediate shaft is partially flexible.

38. The device of claim 31, wherein the intermediate shaft comprises a PTFE polymer.

39. The device of claim 31, wherein the intermediate shaft comprises plastic.

40. The device of claim 31, wherein the intermediate shaft comprises bushings or bearings.

41. The device of claim 31, wherein the intermediate shaft comprises one or more flat portions configured to facilitate reception by the outer sleeve.

42. The device of claim 31, wherein the intermediate shaft comprises openings configured to allow for complete sterilization.

43. The device of claim 31, wherein the intermediate shaft comprises a throughslot configured to allow the diameter of the intermediate sleeve to expand slightly when receiving the reamer shaft.

44. The device of claim 43, wherein the outer sleeve constricts a length of the intermediate sleeve tightly against the reamer shaft.

45. The device of claim 43, wherein the outer sleeve constricts the intermediate sleeve tightly against the reamer shaft along specific bearing locations.
